(19) United States
(12) Patent Application Publication
(10) Pub. No.: US 2008/0275122 A1
(43) Pub. Date: Nov. 6, 2008

(54) N-HALOGENATED AMINO ACID FORMULATIONS WITH ANTI-INFLAMMATORY COMPOUNDS

(75) Inventors: Masood A. Chowhan, Arlington, TX (US); Wesley Wehsin Han, Arlington, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/112,352

(22) Filed: Apr. 30, 2008

Related U.S. Application Data
(60) Provisional application No. 60/915,277, filed on May 1, 2007.

(51) Int. Cl. A61K 31/195; A61P 37/08; A61P 29/00

(52) U.S. Cl. 514/561

(57) ABSTRACT

The present invention relates to a formulation having antimicrobial activity comprising a N-halogenated amino acid and a anti-inflammatory compound. This specification also describes methods for treating a tissue infection comprising contacting the infected tissue with a pharmaceutically effective amount of a formulation comprising a N-halogenated amino acid and a anti-inflammatory compound. This specification further describes a method for treating respiratory infections comprising contacting the site of the respiratory infection with a pharmaceutically effective amount of a formulation comprising a N-halogenated amino acid and a anti-inflammatory compound.

### CROSS-REFERENCE TO RELATED APPLICATION

This application claims priority under 35 U.S.C. §119 to U.S. Provisional Patent Application No. 60/915,277 filed May 1, 2007, the entire contents of which are incorporated herein by reference.

### TECHNICAL FIELD OF THE INVENTION

The present invention generally relates to methods for treating microbial infections using formulations comprising N-halogenated amino acids and anti-inflammatory compounds. The present invention further relates to N-halogenated amino acid-containing formulations comprising anti-inflammatory compounds.

### BACKGROUND OF THE INVENTION

It is generally desirable to use the minimum quantity of an antimicrobial compound necessary to achieve desired effects. This is because undesirable side-effects are more probable when higher concentrations of an antimicrobial are used at a delivery site through the use of, for example, high concentration formulations, more frequent dosing, or longer-duration treatment. Unfortunately, while the use of lower concentrations of antimicrobial compounds generally helps to reduce the potential for undesirable effects, this practice increases the risk that the antimicrobial may not achieve the required level of anti-infective effect. Also, microbial resistance can also develop quickly if antimicrobial compounds are not used at a sufficient concentration. Therefore, inventions that improve the activity of antimicrobial compounds are desirable as they allow for decreased concentrations of such compounds to be used at a delivery site, reducing the incidence and risk of undesired side effects and microbial resistance.

N-halogenated amino acid compounds are known to have desirable antimicrobial properties including antibacterial, anti-infective, antifungal, and/or antiviral properties. Many such N-halogenated amino acid compounds are disclosed in U.S. Patent Application Publication Nos. 2005/0065115 and 2006/0247209, the entire contents of which are incorporated by reference herein.

To cite one of many applications, the use of formulations having antimicrobial properties is important for the treatment of ophthalmic infections such as conjunctivitis. Conjunctivitis can be caused by various kinds of microbes, with most cases being due to bacteria and/or viruses. Unfortunately, conjunctivitis symptoms are not specific to the etiology of the infectious agent and significant testing may be required to determine the causative agent or microbe. Viral conjunctivitis, often caused by adenovirus, is highly contagious yet has no currently known efficacious treatment that provides other than symptom relief. Finally, care must be taken in selecting appropriate agents for treating conjunctivitis, given the sensitive tissues affected by the infection. In view of the above-recited difficulties in treatment, formulations for treating conjunctivitis are needed that have broad-spectrum antimicrobial properties capable of treating bacteria, viruses, fungi, etc., a benign toxicological profile, and/or characteristics that prevent the transmission of contagious infectious agents.

Microbial resistance to conventional antimicrobial treatment is an ongoing concern to medical professionals. Until the problem of resistance is overcome, a steady supply of new treatments and therapies for treating microbial infections is required in order to blunt the effect of microbe mutations that render conventional therapies less effective or, in certain cases, ineffective.

Additionally, microbial infection of tissues often results in serious inflammation that can impede or prevent treatment of the infection and subsequent tissue healing. Also, many of the symptoms produced by infection are often caused by tissue inflammation. Thus, control of inflammation is an important consideration when treating microbial infection. The use of minimum quantities of antimicrobial compounds, while effective to kill or inhibit microbes causing infections, may not be sufficient to reduce inflammation, or may not be efficacious when tissue is inflamed. Treatments for microbial infection are therefore needed that also resolve tissue inflammation.

### BRIEF SUMMARY OF THE INVENTION

The present invention relates to methods for treating infected tissues with formulations comprising a N-halogenated amino acid and a anti-inflammatory compound.

The present invention further relates to N-halogenated amino acid-comprising formulations further comprising an anti-inflammatory compound. These formulations comprise a N-halogenated amino acid such as, for example, 2,2-dimethyl-N,N-dichlorotaurine and an anti-inflammatory compound such as a non-steroidal anti-inflammatory compound or an anti-allergy compound.

Yet another embodiment of the present invention is a method for treating respiratory infections comprising contacting the site of the respiratory infection with a formulation comprising a N-halogenated amino acid and a anti-inflammatory compound.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### DETAILED DESCRIPTION OF THE INVENTION

### I. Definitions

Unless defined otherwise, technical and scientific terms used herein have the same meaning as commonly understood by one of ordinary skill in the art.

As used herein, the term “anti-inflammatory” refers to an ability to counteract, prevent, and/or reduce tissue inflammation caused by infection, disease, and/or trauma. Anti-inflammatory compounds of the present invention possess such ability.

As used herein, the term “antimicrobial” refers to an ability to kill, inhibit, and/or prevent the growth of microbes (to include, without limitation, bacterial, viruses, yeast, fungi, spores, protozoa, parasites, etc.), or to attenuate and/or eradicate a microbial infection. Antimicrobial compounds of the present invention posses such ability.

As used herein, the term “subject” refers to either a human or to non-human domesticated or non-domesticated animals (such as primates, mammals, vertebrates, invertebrates, etc.). The terms “subject” and “patient” may be used interchangeably herein.

As used herein, the terms “treatment”, “treating”, and the like mean obtaining a desired pharmacologic and/or physiologic effect. The desired effect may be, without limitation, prevention of a disease or infection in certain usage and/or may be therapeutic in terms of a partial or complete cure for a disease or infection and/or adverse effect attributable to the disease or infection.

### II. Methods and Formulations

The formulations of the present invention comprise an N-halogenated amino acid and an anti-inflammatory compound. The N-halogenated amino acids of the present invention have the following general formula:

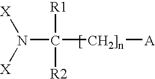

where X is one or more halogens and R1 and R2 are any of the nonpolar, uncharged polar, and charged polar amino acid and amino acid derivative side chains known to those of skill in the art. A represents an acid such as a carboxylic, sulfonic, phosphoric, boric or other acid known to those of skill in the art. There may be one or more carbon atoms between the amine and acid, and each carbon may contain on or more R substituents.

The preferred N-halogenated amino acids of the present invention have the following structure: haloamino-stabilizer-linker-acid, where (a) the “haloamino” is either N-halogen or N,N-dihalogen (e.g., —NHCl or —NCl₂); (b) the “stabilizer” comprises sidechains attached to the carbon next to the haloamino group (e.g., hydrogen, —CH₃, lower alkyl, the group —COOH or a C₃₋₆ cycloalkyl ring); (3) the “linker” is either alkyl or cycloalkyl; and (d) the “acid” is one of the following: —COOH, —SO₃H, —P(═O)(OH)₂, —B(OH)₂ or hydrogen, and all the pharmaceutically acceptable salts of these acids generally known to those skilled in the art, including but not limited to sodium, potassium, calcium, etc.

The most preferred N-halogenated amino acids are 2,2-dimethyl-N,N-dichlorotaurine, analogs of 2,2-dimethyl-N,N-dichlorotaurine formed by replacement of the sulfonic acid group with carboxylic acid, phosphoric acid, borate, etc., 2,2-di alkyl-N,N-dichlorotaurine or 2,2-R—N,N-dichlorotaurine, where R is an aliphatic or aromatic side chain. Methyl groups of N-halogenated amino acids may be replaced with alkyl, aryl, benzyl, or other hydrocarbon cyclic or non-cyclic groups.

Anti-inflammatory compounds include, but are not limited to, non-steroidal anti-inflammatory drugs (NSAIDs). NSAIDs that may be used in embodiments of the present invention include all non-commercially and commercially available NSAIDs suitable for use in a subject such as ibuprofen, naproxen, nabumetone, ketorolac, sulindac, diclofenac, nepafenac, and cyclooxygenase-2 inhibitors (COX-2 inhibitors). The NSAIDs described herein also include the pro-drugs which are metabolized to their parent NSAIDs upon entering the body. Preferred NSAIDs are amfenac, nepafenac, ketorolac, diclofenac, and flurbiprofen, and other NSAID compounds as disclosed in U.S. Pat. Nos. 5,475,034 and 4,910,225, both of which are incorporated herein by reference.

Yet other examples of anti-inflammatory compounds are anti-allergic and anti-itching agents such as mast cell stabilizers, including but not limited to olopatadine, ketotifen, nedocromil sodium, cromolyn sodium, and antihistamines.

Certain methods and formulations of the present invention comprise the use of N-halogenated amino acids with phase transfer agents to improve their antimicrobial properties. U.S. Provisional Patent Application No. 60/915,291, filed May 1, 2007, entitled “N-HALOGENATED AMINO ACID FORMULATIONS,” herein incorporated by reference in its entirety, discloses such N-halogenated amino acid formulations.

### III. Applications

The invention is particularly directed toward treating subjects suffering from infected tissue, particularly mammalian subjects infected with any infectious bacteria or fungi, and more particularly in a human mammal. Other bacterial infections and protozoal infections and disorders related to such infections that may be treated or prevented in accord with the method of the present invention are referred to in J. P. Sanford et al., “The Sanford Guide to Antimicrobial Therapy 2007” 37th Edition, (Antimicrobial Therapy, Inc.). The present invention is also particularly directed to antimicrobial formulations for and methods of treating ophthalmic, otic, dermal, upper respiratory, lung/lower respiratory, esophageal, and nasal/sinus infections.

Certain embodiments of the present invention are particularly useful for treating ophthalmic tissue infections. Examples of ophthalmic infections that may be treated using formulations and methods of the present invention include conjunctivitis, keratitis, blepharitis, dacyrocystitis, hordeolum and corneal ulcers. The methods and formulations of the invention may also be used prophylactically in various ophthalmic surgical procedures that create a risk of infection.

Otic and nasal/sinus tissue infections may also be treated by embodiments of the present invention. Examples of otic conditions that may be treated with formulations and methods of the present invention include otitis externa and otitis media, including those situations where the tympanic membrane has ruptured or tympanostomy tubes have been implanted. Examples of nasal/sinus infections that may be treated with formulations and methods of the present invention include rhinitis, sinusitis, nasal carriage and situations where the nasal or sinus tissues are affected by surgery. Examples of respiratory infections and infectious agents include pneumonia, influenza, bronchitis, respiratory syncytial virus, etc.

Other embodiments of the present invention may also be used in treatment solutions for skin and body tissue surfaces of a subject, providing antimicrobial activity against bacteria, fungi, viruses, protozoa, etc. Such treatment may be used to treat infected body tissue or wounds having one or more varieties of infectious agents present. These embodiments may also be used for treating the dermatological diseases caused by bacteria, fungi, viruses, protozoa, etc, Such embodiments may comprise formulations having one or more N-halogenated amino acids and anti-inflammatory compounds in a solution that is in a vehicle suitable for topical use.

Certain embodiments of the present invention may be used for treating onychomycosis. Onychomycosis refers to the invasion of a nail plate by a fungus. The infection may be due to a dermatophyte, yeast, or nondermatophyte mold. The term “tinea unguium” is used specifically to describe invasive dermatophytic onychomycosis. Implicated dermatophytes include, but are not limited to: Epidermophyton floccosum, Microsporum audouinii, Microsporum canis, Microsporum gypseum, Trichophyton mentagrophytes, Trichophyton rubrum, Trichophyton schoenleinii, Trichophyton tonsurans. Additional fungi that may cause onychomycosis include, but are not limited to, Acremonium spp., Aspergillus spp., Candida spp., Fusarium oxysporum, Scopulariopsis brevicaulis, Onychocola canadensis, and Scytalidium dimidiatum.

Embodiments of the present invention may also be used prophylactically to prevent infection of a tissue by an infectious agent. In such embodiments, a tissue at risk of infection is contacted with a formulation of the present invention.

### IV. Pharmaceutics and Formulations

A. Dosage

The phrase “pharmaceutically effective amount” is an art-recognized term, and refers to an amount of a compound that, when incorporated into a pharmaceutical formulation of the present invention, produces some desired effect at a reasonable benefit/risk ratio applicable to any medical treatment. The effective amount may vary depending on such factors as the disease or infectious agent being treated and the amount of inflammation, the particular formulation being administered, or the severity of the disease or infection agent.

The phrase “pharmaceutically acceptable” is art-recognized and refers to formulations, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art.

In particular embodiments, a formulation is administered once a day. However, the formulations of the present invention may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication. Factors involved in this determination include the disease to be treated, particular characteristics of the subject, and the particular antimicrobial formulation.

B. Formulations

In addition to the N-halogenated amino acid and the anti-inflammatory compound, the formulations of the present invention optionally comprise one or more excipients. Excipients commonly used in pharmaceutical formulations include, but are not limited to, tonicity agents, preservatives, chelating agents, buffering agents, surfactants and antioxidants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, and mixtures of those polymers. The concentration of the excipient is, typically, from 1 to 100,000 times the concentration of the N-halogenated amino acid. In preferred embodiments, excipients are selected on the basis of their inertness towards the N-halogenated amino acid and anti-inflammatory compound.

Suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, include ionic and nonionic surfactants, though nonionic surfactants are preferred, RLM 100, POE 20 cetylstearyl ethers such as Procol® CS20 and poloxamers such as Pluronic® F68. Suitable antioxidants include, but are not limited to, sulfites, ascorbates, butylated hydroxyanisole (BHA) and butylated hydroxytoluene (BHT).

In particular embodiments, formulations are suitable for application to mammalian eyes. For example, for ophthalmic administration, the formulation may be a solution, a suspension, a gel, or an ointment.

For use in sinus and respiratory infection applications, formulations may be used that are suitable for aerosol formation using nebulizers or other such devices well known to those of skill in the art.

In preferred aspects, embodiments of the present invention will be formulated for topical application to the eye in aqueous solution in the form of drops. The term “aqueous” typically denotes an aqueous formulation wherein the excipient is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or a similar vehicle, or as dissolvable inserts that are placed beneath the eyelids. In yet another aspect, components of the invention may be delivered to the eye as ointment, water-in-oil and oil-in-water emulsions.

For topical ophthalmic applications, the formulations are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The pH of the solution may be in an ophthalmic acceptable range of 3.0 to 8.0. The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

The formulations set forth herein may comprise one or more preservatives. Examples of such preservatives include p-hydroxybenzoic acid ester, alkyl-mercury salts of thiosalicylic acid, such as thiomersal, phenylmercuric nitrate, phenylmercuric acetate, phenylmercuric borate, sodium perborate, sodium chlorite, parabens such as methylparaben or propylparaben, alcohols such as chlorobutanol, benzyl alcohol or phenyl ethanol, guanidine derivatives such as polyhexamethylene biguanide, sodium perborate, or sorbic acid. In certain embodiments, the formulation may be self-preserved that no preservation agent is required.

In certain embodiments, the N-halogenated amino acid and the anti-inflammatory compound are in a formulation that comprises one or more tear substitutes. A variety of tear substitutes are known in the art and include, but are not limited to: monomeric polyols, such as glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain formulations of the present invention may be used with contact lenses or other ophthalmic products.

In some ophthalmic embodiments, the formulations set forth herein have a viscosity of 0.5-100 cps, preferably 0.5-50 cps, and most preferably 1-20 cps. This relatively low viscosity insures that the product is comfortable, does not cause blurring, and is easily processed during manufacturing, transfer and filling operations.

The N-halogenated amino acids and anti-inflammatory compounds described herein may be included in various types of formulations having activities in addition to antimicrobial activity. Examples of such formulations include: ophthalmic pharmaceutical formulations (such as ocular lubricating products and artificial tears), astringents, topical disinfectants (alone or in combination with other antimicrobial agents such as, for example, betadine, etc.) and so on.

To effectively treat various microbial infections and to minimize side-effects, the antimicrobial activity of a formulation should be maximized so that a minimum amount of active ingredient is used. The activity of the antimicrobial formulations of the present invention is the result of the antimicrobial agent itself; the formulation components other than the N-halogenated amino acid normally cause little effect. The amount of the N-halogenated amino acid in particular formulations can be determined by persons skilled in the art. The concentration required to achieve the desired antimicrobial activity while retaining acceptable safety and toxicity properties is referred to herein as “an effective amount”.

In order to reduce the tissue inflammation associated with microbial infection, the anti-inflammatory activity of the formulation should be maximized so that a minimum amount of active ingredient is used. The amount of the anti-inflammatory compound in particular formulations can be determined by persons skilled in the art. The concentration required will depend on the particular anti-inflammatory compound selected, the presence or absence of other ingredients that have anti-inflammatory activity, and the function of the anti-inflammatory agents contained in the formulations. The concentration required to achieve the desired anti-inflammatory activity while retaining acceptable safety and toxicity properties is referred to herein as “an effective amount”.

It is contemplated that the concentrations of the ingredients comprising the formulations of the present invention can vary. In non-limiting aspects, the percentage can be calculated by weight or volume of the total formulation. A person of ordinary skill in the art would understand that the concentrations can vary depending on the addition, substitution, and/or subtraction of ingredients in a given formulation.

In certain embodiments, topical formulations (particularly topical ophthalmic formulations, as noted above) are preferred which have a physiological pH matching the tissue to which the formulation will be applied or dispensed.

Certain formulations of the present invention can be administered in a two-part system. For instance, the N-halogenated amino acid can be present in one part of the formulation and one or more components of the formulation, such as an anti-inflammatory compound, are separated in a separate container or different portion of the same container until a user is ready to administer the formulation. At the instant of administration or before, the two parts may be mixed by a user. The two-part system may be useful in cases where one or more components of the formulation have stability problems when combined. Also, a two-part system may be utilized as part of a nasal/sinus spray dispensing system in certain embodiments.

C. Route of Administration

In the methods set forth herein, administration to a subject of a pharmaceutically effective amount of a formulation that comprises an N-halogenated amino acid and a anti-inflammatory compound may be by any method known to those of ordinary skill in the art.

For example, the formulation may be administered locally, topically, intradermally, intralesionally, intranasally, subcutaneously, orally, by inhalation, by injection, by localized perfusion bathing target cells directly, via a catheter, or via lavage.

In particular embodiments, the formulation is administered topically to the ocular surface. Regarding ophthalmic administration, it is contemplated that all local routes to the eye may be used, including topical, subconjunctival, periocular, retrobulbar, subtenon, intraocular, subretinal, posterior juxtascleral, and suprachoroidal administration.

Various otic administration techniques are also contemplated. In particular embodiments, the formulation may be delivered directly to the ear canal (for example: topical otic drops or ointments; slow release devices in the ear or implanted adjacent to the ear). Local administration includes otic intramuscular, intratympanic cavity and intracochlear injection routes of administration for the formulations. It is further contemplated that certain formulations of the invention may be formulated in intraotic insert or implant devices. For instance, delivery of the formulations can be accomplished by endoscopic assisted (including laser-assisted endoscopy to make the incision into the tympanic membrane) injection into the tympanic cavity as set forth, for example, in Amer. J. Otology, Vol. 16:158-163, 1995; Ear Nose Throat, Vol. 76:674-678, 1997; Otolarngol Head Neck Surg., Vol. 120:649-655, 1999. Local administration can also be achieved by injection through the tympanic membrane using a fine (EMG recording) needle, through use of an indwelling catheter placed through a myringotomy incision, and injection or infusion through the Eustachian tube by means of a small tubal catheter. Furthermore, the formulations can be administered to the inner ear by placement of gelfoam or a similar absorbent and adherent product soaked with the formulations, against the window membrane of the middle/inner ear or adjacent structure with due discretion and caution by a skilled clinician.

Administration of the formulations described herein for the treatment of sinus tissue infection, nasal infection, upper respiratory infection, lung/lower respiratory infection, esophageal infection, and the various combinations can be via a number of methods known to those of skill in the art. Preferred administration for lower respiratory infections is via aerosol formation by use of a nebulizer or other similar device. Formulations for the treatment of sinus infections can be administered in droplet form (often otic formulations can be used for the treatment of sinus infections) or by aerosol formation. Esophageal infections may be treated by administration of a liquid or aerosol formulation.

Other modes of administration of the formulations of the present invention are via skin patches, intrapulmonary, intranasally, via liposomes formulated in an optimal manner, and via slow release depot formulations. Various devices can be used to deliver the formulations to the affected ear compartment; for example, via catheter or as exemplified in U.S. Pat. No. 5,476,446 which provides a multi-functional apparatus specifically designed for use in treating and/or diagnosing the inner ear of the human subject. Also see U.S. Pat. No. 6,653,279 for other devices for this purpose.

### V. Examples

The following examples are presented to further illustrate selected embodiments of the present invention. To these formulations (and to other embodiments of the present invention) a viscosity agent such as hydroxypropylmethylcellulose (HPMC), sodium carboxymethylcellulose (NaCMC), hydroxyethylcellulose (HEC), xanthan gum, etc., can be incorporated. Such formulations according to embodiments of the present invention may be produced in a variety of material states such as, for example, solid, liquid, semi-solid, etc.

### Example 1

| Ingredient | % w/v |
|---|---|
| Sodium 2,2-dimethyl-N,N-dichlorotaurine | 0.1% |
| Nepafenac | 0.1% |
| Sodium Chloride | 0.8% |
| Hydrochloric Acid | q.s. pH 4 |
| Sodium Hydroxide | q.s. pH 4 |
| Purified Water | q.s. 100% |

### Example 2

| Ingredient | % w/v |
|---|---|
| Sodium 2,2-dimethyl-N,N-dichlorotaurine | 0.1% |
| Diclofenac | 0.05% |
| Sodium Chloride | 0.8% |
| Hydrochloric Acid | q.s. pH 5.5 |
| Sodium Hydroxide | q.s. pH 5.5 |
| Purified Water | q.s. 100% |

### Example 3

The antimicrobial activity of N-halogenated amino acid formulations were evaluated by a standard microbiological analysis. The results of this evaluation are summarized in Tables 1 and 2 below. For the evaluation, bacterial and fungal isolates were grown overnight on appropriate agar media as source of fresh cells. A suspension of these fresh cells was prepared in saline at approximately 1×10⁸ cfu/mL. These suspensions were added directly to the test agents (various solutions of 2,2-dimethyl-N,N-dichlorotaurine and control solutions). The initial concentration of cells in the test agent solutions was approximately 1×10⁶ cfu/mL. The exposure of microorganisms to the test agent was conducted at room temperature for up to 60 minutes. At selected times, an aliquot was withdrawn and diluted into phosphate buffered saline at 4° C. Viability was determined following serial dilution and filtration onto Milliflex cassettes.

TABLE 1
Evaluation with S. aureus

| | Sampling | | | | | |
|---|---|---|---|---|---|---|
| | Time | # | Correction | Dilution | Viable | % |
| Product | (min) | Colonies | Factor | Factor | Cells/ml | Survivors |
| | 0 | 103 | 1.11 | 10000 | 1143300 | 100.00 |
| Control (water) | 5 | 91 | 1.23 | 10000 | 1119300 | 97.90 |
| | 15 | 108 | 1.23 | 10000 | 1328400 | 116.19 |
| | 60 | 102 | 1.23 | 10000 | 1254600 | 109.73 |
| | 0 | 128 | 1.11 | 10000 | 1420800 | 100.00 |
| Vehicle | 60 | 79 | 1.23 | 10000 | 971700 | 68.3910 |
| pH 4.0 w/sodium | | | | | | |
| acetate | | | | | | |
| | 180 | 88 | 1.23 | 10000 | 1082400 | 76.1824 |
| | 1440 | 101 | 1.23 | 10000 | 1242300 | 87.4367 |
| | 0 | 128 | 1.11 | 10000 | 1420800 | 100.00 |
| 2,2-dimethyl-N,N- | 5 | 21 | 1.11 | 1 | 23.31 | 0.001641 |
| dichlorotaurine | | | | | | |
| 0.001% | | | | | | |
| pH 4.0 w/sodium | | | | | | |
| acetate | | | | | | |
| | 15 | 1 | 1.11 | 1 | 1.11 | 0.000078 |
| | 60 | 0 | 1.11 | 1 | 0 | 0.000000 |
| | 0 | 98 | 1.11 | 10000 | 1087800 | 100.00 |
| 2,2-dimethyl-N,N- | 5 | 74 | 1.23 | 10 | 910.2 | 0.084 |
| dichlorotaurine | | | | | | |
| 0.001% | | | | | | |
| pH 4.0 w/adipic acid | | | | | | |
| | 15 | 210 | 1.11 | 1 | 233.1 | 0.021 |
| | 60 | 2 | 1.11 | 1 | 2.22 | 0.0002 |

As shown above in Table 1, all formulations comprising N-halogenated amino acid possessed significant antimicrobial properties against S. aureus relative to control formulations.

TABLE 2
Evaluation with C. albicans

| | Sampling | | | | | |
|---|---|---|---|---|---|---|
| | Time | # | Correction | Dilution | Viable | % |
| Product | (min) | Colonies | Factor | Factor | Cells/ml | Survivors |
| | 0 | 44 | 1.11 | 10000 | 488400 | 100.00 |
| Control (water) | 5 | 59 | 1.11 | 10000 | 654900 | 134.09 |
| | 15 | 59 | 1.11 | 10000 | 654900 | 134.09 |
| | 60 | 51 | 1.11 | 10000 | 566100 | 115.91 |
| | 0 | 52 | 1.11 | 10000 | 577200 | 100.000 |
| Vehicle | 5 | 58 | 1.11 | 10000 | 643800 | 111.538 |
| pH 4.0 w/sodium acetate | | | | | | |
| | 15 | 58 | 1.11 | 10000 | 643800 | 111.538 |
| | 60 | 59 | 1.11 | 10000 | 654900 | 113.462 |
| | 0 | 56 | 1.11 | 10000 | 621600 | 100.0000 |
| 2,2-dimethyl-N,N- | 5 | 40 | 1.11 | 10000 | 444000 | 71.4286 |
| dichlorotaurine 0.001% | | | | | | |
| pH 4.0 w/sodium acetate | | | | | | |
| | 15 | 71 | 1.11 | 1000 | 78810 | 12.6786 |
| | 60 | 22 | 1.11 | 100 | 2442 | 0.3929 |

As shown above in Table 2, all formulations comprising N-halogenated amino acid possessed significant antimicrobial properties against C. albicans relative to control formulations.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A formulation having antimicrobial activity comprising a N-halogenated amino acid and a anti-inflammatory compound.

2. The formulation of claim 1 wherein the anti-inflammatory compound is selected from the group consisting of:
NSAIDs, antiallergic agents, and combinations thereof.

3. The formulation of claim 1 wherein the N-halogenated amino acid is a chlorotaurine.

4. The formulation of claim 3 wherein the chlorotaurine is sodium 2,2-dimethyl-N,N-dichlorotaurine.

5. The formulation of claim 3 wherein the N-halogenated amino acid forms an ion-pair with a component of said formulation.

6. A method for treating a tissue infection comprising:
contacting the infected tissue with a pharmaceutically effective amount of a formulation comprising a N-halogenated amino acid and a anti-inflammatory compound.

7. The method of claim 6 wherein the anti-inflammatory compound is selected from the group consisting of:
NSAIDs, antiallergic agents, and combinations thereof.

8. The method of claim 6 wherein the N-halogenated amino acid is a chlorotaurine.

9. The method of claim 8 wherein the chlorotaurine is sodium 2,2-dimethyl-N,N-dichlorotaurine.

10. The method of claim 6 wherein said infected tissue is ocular, otic, nasal, sinus, or dermal tissue.

11. The method of claim 6 wherein said formulation is a two-part formulation.

12. A method for treating respiratory infections comprising:
contacting the site of the respiratory infection with a pharmaceutically effective amount of a formulation comprising a N-halogenated amino acid and a anti-inflammatory compound.

13. The method of claim 12 where the respiratory infection is selected from the group consisting of:
sinus tissue infection, nasal infection, upper respiratory infection, lung/lower respiratory infection, esophageal infection, and combinations thereof.

14. A method for preventing tissue infection comprising:
contacting a tissue at risk for infection with a pharmaceutically effective amount of a formulation comprising a N-halogenated amino acid and a anti-inflammatory compound.
